(19) United States
(12) Patent Application Publication
(10) Pub. No.: US 2026/0131198 A1
(43) Pub. Date: May 14, 2026

(54) FITNESS TRACKING SYSTEM AND METHOD OF OPERATING THE SAME

(71) Applicant: Train Fitness Inc., Toronto (CA)

(72) Inventors: Robert Andrew JUST, Toronto (CA); Vivek VERMA, Delhi (IN)

(21) Appl. No.: 19/389,807

(22) Filed: Nov. 14, 2025

Related U.S. Application Data
(60) Provisional application No. 63/720,351, filed on Nov. 14, 2024.

(51) Int. Cl. A63B 24/00

(52) U.S. Cl. CPC A63B 24/0062; A63B 24/0006; A63B 2220/40; A63B 2220/836

(57) ABSTRACT

A fitness tracking system can include one or more wearable devices comprising sensors configured to detect motion of a user and a computing device communicatively coupled to the wearable devices. The computing device can include at least one processor and non-transitory memory storing instructions which, when executed by the at least one processor, cause the at least one processor to perform operations including receiving, from the one or more wearable devices, first data characterizing raw motion of the user, determining, using an artificial intelligence (AI) model, one or more physiological characteristics of the user, predicting, using the AI model, based on the one or more physiological characteristics and the first data, an exercise activity being performed by the user, and providing data characterizing the predicted exercise activity.

### CROSS-REFERENCE TO RELATED APPLICATIONS

This application claims the benefit of U.S. Provisional Patent Application No. 63/720,351, filed on November 14, 2024, entitled “Fitness Tracking System and Method of Operating the Same,” the entirety of which is hereby incorporated by reference.

### FIELD

Embodiments of the present disclosure generally relate to fitness tracking systems.

### BACKGROUND

Fitness tracking systems are designed to monitor, record, and analyze physical activity to support users in achieving their health and fitness goals. These systems provide insights into exercise habits, help track progress over time, and encourage consistent engagement with fitness routines.

Existing fitness tracking systems typically include a mobile computing device, such as a smart phone, which serves as a central hub for collecting and managing exercise data. Users often manually input workout-related information into the mobile device, including the types of exercises performed, the number of repetitions, and the amount of weight used. In addition to manual data entry, some fitness tracking systems incorporate wearable computing devices, such as smart watches or fitness tracking bands. These wearable devices collect motion data while the user is exercising and enable the tracking of physiological parameters, such as heart rate, in real time during physical activity.

### SUMMARY

The present disclosure describes fitness tracking systems and methods of operating the same. In some embodiments, the fitness tracking systems are configured to conduct operations of a machine learning model for automatically generating exercise activity predictions, associated activity repetition counts, exercise activity form analytics, or exercise sequence recommendations, among other feedback signals for a user. The exercise activity predictions and associated activity repetition counts may be based on time-series sensor data from one or more wearable computing devices.

In some embodiments, wearable computing devices for generating time-series sensor data representing user movement may include smart watch devices, audio devices (e.g., wireless ear buds), smart garments, fitness tracking bands, among other examples.

To illustrate, embodiments of the present disclosure may be configured for distinguishing between two or more similar but nonetheless different exercise activities. For example, a user doing bench press exercises with a barbell may exert similar physiological motion of the upper body as a user doing bench press exercises with dumbbells. Accordingly, embodiments of the present disclosure provide devices for generating exercise activity predictions with increased precision or granularity, thereby being able to increase accuracy when distinguishing exercise activities having common physiological motion characteristics but nonetheless being different exercise activities.

In one aspect, systems for fitness tracking are described. In some embodiments, a fitness tracking system can include at least one processor and non-transitory memory storing instructions which, when executed by the at least one processor, cause the at least one processor to perform the following operations. First data characterizing raw motion of a user can be received from one or more wearable devices having one or more sensors configured to detect motion of a user. Using an artificial intelligence (AI) model, one or more physiological characteristics of the user can be determined. Based on the one or more physiological characteristics and the first data, the AI model can be used to predict an exercise activity being performed by the user. Data characterizing the predicted exercise activity can then be provided.

In some embodiments, the one or more wearable devices include a smart watch, a fitness tracking band, wireless headphones, or wireless earphones.

In some embodiments, the first data can include a time series of acceleration vectors representing a magnitude and direction of an acceleration of a body part of the user.

In some embodiments, the AI model can include one or more convolutional neural network (CNN) layers, one or more long short-term memory (LSTM) layers, one or more embedding layers, or a combination thereof.

In some embodiments, the operations can further include using the AI model to transform the first data into second data representing one or more motion features. Transforming the first data into the second data can include extracting the one or more motion features from the first data by one or more convolutional neural networks (CNNs) of the AI model.

In some embodiments, the operations can further include encoding, by one or more embedding layers of the AI model, the one or more physiological characteristics as one or more values.

In some embodiments, the first data can include data characterizing raw motion of the user during a first time period prior to performing an exercise movement, data characterizing raw motion of the user during a second time period in which the exercise movement is performed, and data characterizing raw motion of the user during a third time period following performance of the exercise movement.

In some embodiments, the operations can further include predicting, by the AI model, one or more metrics associated with the exercise activity. The one or more metrics can include a first value corresponding to a number of repetitions of the exercise activity and a second value quantifying an amount of weight or resistance used during the exercise activity.

In some embodiments, predicting the exercise activity performed by the user can include identifying a first type of motion based on a first subset of the first data, identifying a second type of motion based on a second subset of the first data, and predicting a compound exercise activity performed by the user. The compound exercise activity can be an exercise activity that includes the first type of motion and the second type of motion.

In some embodiments, the operations can further include receiving data characterizing an environment of the user. Predicting the exercise activity can be further based on the data characterizing the environment of the user. The data characterizing the environment of the user can include data characterizing an audible noise, a magnetic field strength, or a wireless network signal in a vicinity of the user. In some embodiments, the operations can further include determining, by the AI model, a location of the user based on the data characterizing the environment of the user, and predicting the exercise activity can be further based on the location of the user.

In another aspect, a non-transitory computer-readable medium or media having stored thereon machine interpretable instructions which, when executed by a processor may cause the processor to perform one or more fitness tracking methods are described.

In various aspects, corresponding systems and devices, and logic structures such as machine-executable coded instruction sets for implementing such systems, devices, and methods are described.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrates a fitness tracking platform, in accordance with embodiments of the present disclosure;

FIG. 2 illustrates a block diagram of a fitness tracking platform, in accordance with embodiments of the present disclosure;

FIG. 3 illustrates an example smart watch device worn by a user partaking in weightlifting exercises, in accordance with embodiments of the present disclosure;

FIG. 4 illustrates a mobile computing device carried by a user in a garment pocket in varying orientations during an exercise activity, in accordance with embodiments of the present disclosure;

FIG. 5 illustrates a block diagram of a wearable computing device, in accordance with embodiments of the present disclosure;

FIG. 6 illustrates a flowchart of an exercise prediction method, in accordance with embodiments of the present disclosure;

FIG. 7 illustrates a flowchart of an exercise prediction method, in accordance with embodiments of the present disclosure;

FIG. 8 illustrates a flowchart of a method of fitness exercise tracking, in accordance with embodiments of the present disclosure;

FIG. 9 illustrates a diagram of an artificial intelligence model implemented by a fitness tracking system, in accordance with embodiments of the disclosure;

FIG. 10 illustrates a flowchart of a method of fitness tracking, in accordance with embodiments of the disclosure; and

FIG. 11 illustrates example views of a graphical user interface (GUI) of a smart watch application associated with a fitness tracking system.

### DETAILED DESCRIPTION

While fitness tracking systems have become increasingly sophisticated, existing implementations remain limited in their ability to automatically identify and analyze specific user movements during exercise, particularly anaerobic exercise. These systems typically rely on manual input from users to record workout-related information, such as the type of exercise performed, the number of repetitions completed, and the amount of weight used. This reliance on manual data entry can be time-consuming and may lead to incomplete or inaccurate records, reducing the overall utility of the system for tracking and analyzing fitness progress.

Some existing fitness tracking systems incorporate wearable computing devices, such as smart watches or fitness tracking bands, which are capable of detecting when a user is engaged in physical activity. These devices may also be able to broadly classify the type of exercise being performed, such as distinguishing between cardiovascular activity and strength training. However, such systems are generally unable to identify specific movements (e.g., squats, bicep curls, or lunges), cannot determine exercise metrics such as the number of repetitions performed, and lack the capability to assess the quality of movement execution. Furthermore, these systems are typically unable to evaluate user effort based on motion data, limiting their ability to provide meaningful feedback or personalized insights into workout performance.

Described herein are embodiments of fitness tracking systems that may leverage a specialized artificial intelligence (AI) model to analyze motion data in real time while a user is exercising. By processing data from one or more user devices, the system can detect the specific type of movement being performed, such as squats or push-ups, and automatically count the number of repetitions completed within a set. In addition to identifying and quantifying movements, the system can assess the quality of movement execution, providing insight into form and technique, and evaluate the user's effort output during each exercise, enabling a more detailed and personalized understanding of workout performance.

Embodiments of the AI model employed by the described systems enable motion data collected from wearable computing devices to be processed in real time. This motion data is often complex and highly variable, reflecting (often subtle) differences in movement patterns across individuals with diverse physiological characteristics such as height, weight, and gender. Accurately identifying specific exercises, counting repetitions, assessing movement quality, and evaluating user effort requires computational analysis that accounts for these variations. The AI model can be implemented with a large number (e.g., millions) of parameters—including nodes, weights, and biases—that enable the model to execute intensive, multi-dimensional computations with high efficiency. As a result, the described systems can rapidly and accurately analyze large and heterogeneous datasets in view of a unique combination of physiological variables, producing exercise-specific insights that cannot be reliably or feasibly generated in real time through human observation or mental calculation alone.

Certain embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting embodiments and that the scope of the present invention is defined solely by the claims. The features illustrated or described in connection with one embodiment may be combined with the features of other embodiments. Such modifications and variations are intended to be included within the scope of the present invention.

Mobile computing devices and wearable computing devices may be carried or worn by users during one or more activities. For example, smartphones may be commonly carried by a user in a garment pocket. Smart watches may be worn by a user throughout the course of a day and, in some situations, while sleeping. Wireless audio devices such as ear buds may be worn while exercising, among other activities.

In some embodiments, such mobile computing devices and wearable computing devices may include one or more sensors configured to monitor motion-related or environment-related data associated with a computing device. In some embodiments, sensors may include accelerometers, gyroscopes, pedometers, magnetometers, or barometers, among other examples.

Embodiments of fitness tracking systems described herein may be configured to obtain sensor data sets for determining motion or environment conditions associated with a computing device. For example, motion may include movement such as tilt, shake, rotation, acceleration, or swing. In some situations, determined motion or environmental conditions may correspond to user input, user movement, or the physical environmental conditions associated with the user of the computing device. In some embodiments, environment conditions may be associated with pre-activity or post-activity movements, 3rd party data sets associated with geolocation data, magnetometer data associated with detecting equipment devices, among other examples to be described in the present disclosure.

Based on one or more of determined user movement or physical environmental conditions, the computing device may be configured to predict or infer a type of activity being undertaken by a user. Features of exercise tracking systems will be described in the present disclosure.

It may be desirable to provide fitness tracking systems configured to predict or infer an exercise activity type with increased precision or confidence levels / scores, thereby being able to increase accuracy when distinguishing exercise activities having common physiological motion characteristics but nonetheless are different exercise activities.

Some embodiments disclosed herein may be based on a user donning a sole or preferred fitness tracking device, such as a smart watch or other computing device band on the user’s limb during exercise activity. The sole or preferred fitness tracking device may be configured to generate exercise predictions, determine exercise repetition counts, among other examples of operations.

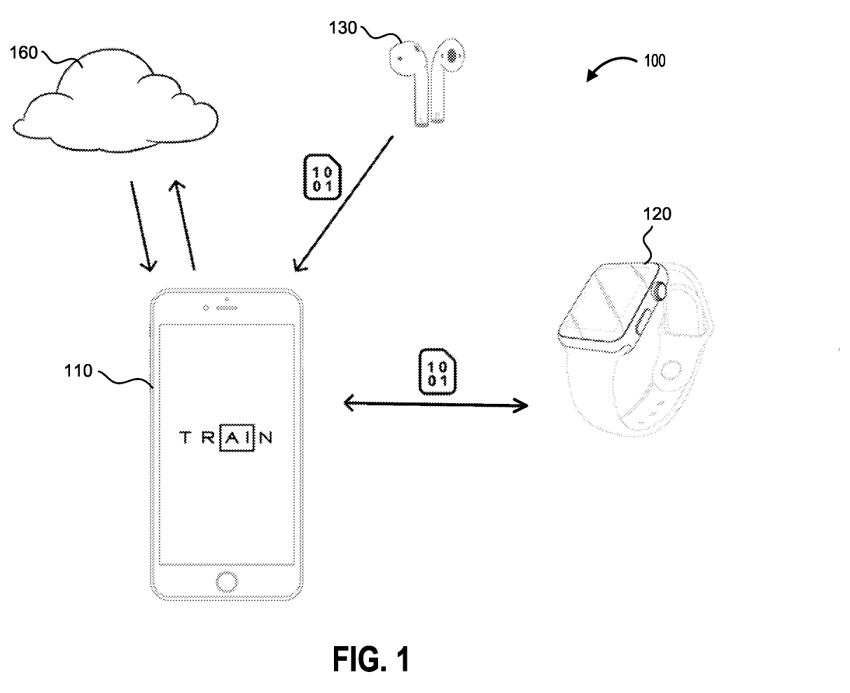

Reference is made to FIG. 1, which illustrates a fitness tracking platform 100, in accordance with an embodiment of the present disclosure. The fitness tracking platform 100 may include a mobile computing device 110. In some embodiments, the mobile computing device 110 may be a smartphone or a pocket personal computer, among other examples, and the mobile computing device 110 may be configured to transmit or receive, via a network, data messages to / from one or more client devices.

In the illustrated embodiment of FIG. 1, the mobile computing device 110 may be configured to conduct operations of machine learning models for generating exercise predictions or determining exercise repetition counts, among other operations, based on sensor data generated at the plurality of other devices of the fitness tracking platform 100. It may be contemplated that operations of machine learning models may be distributed, solely or in part, to other devices of the fitness tracking platform 100.

In some embodiments, client devices may include a smartwatch device 120, an audio device 130, or other wearable computing devices, such as fitness tracking bands, smart eyewear, among other examples. In FIG. 1, two example client devices may be the smartwatch device 120 and a pair of earbud-type audio devices 130. In some other embodiments, the fitness tracking platform 100 may include a single client device or may include any other number of client devices.

In some embodiments, the fitness tracking platform 100 may be configured to transmit or receive, via the network, data messages to and from a data server 160. In some embodiments, the data server 160 may be a centralized application server, Software as a Service (SaaS) computing platform, among other examples.

As will be described with reference to examples in the present disclosure, the data server 160 may be configured with operations to manage features of the fitness tracking platform 100, to provide social media-based functionality for a plurality of users, or to provide distributed computing operations for machine learning models for predicting or inferring types of activity based on data sets representing user movement or physical environmental conditions corresponding to the user. The data server 160 may be configured with other operations. In some embodiments, the distributed computing operations may provide the supplemental features as an add on and where a plurality of features described herein may be executed by the wearable computing device or mobile computing device as a standalone device.

Embodiments of the fitness tracking system 100 may include machine learning or artificial intelligence models for generating predictions of type of user activity and for determining exercise activity statistics to provide feedback to the user. The machine learning models may be trained by training data sets prepared based on sensor data sets associated with video footage of users partaking in exercise activities.

For example, training data sets may be generated by obtaining sensor data from a smart watch, and simultaneously recording and associating video footage of a user conducting exercises (e.g., running, bench presses, pushups, rowing machine exercises, etc.). To illustrate, the sensor data may represent physiological user motion based on gyroscope sensor data and/or accelerometer sensor data recorded at a rate of up to 800 samples per second. Other sensor data sampling rates may be used.

In some embodiments, operations of the machine learning models for generating predictions and for generating exercise activity statistics may be conducted at the mobile computing device 110, at the data server 160, or a combination of devices.

In some embodiments, the training data sets may be augmented or altered for performing feature engineering and to train the machine learning models. For example, subsets of obtained sensor data may be altered to simulate potential exercise behaviors of fitness enthusiasts. Feature engineering operations may include increasing the speed at the front end of an exercise activity set or decreasing the speed at the back end of an exercise activity set to simulate explosive activity repetitions and fatigue, respectively. In some other examples, feature engineering operations may include operations to rotate or transform sensor data signals to simulate different user body types, body builds, among other user characteristics. In some embodiments, video data sets may be associated with one or more flags or time-associated metadata for supporting operations including form analysis or range of motion analysis.

In some embodiments, the machine learning models may be configured to detect exercise activity types when the exercise activity begins or when the exercise activity ceases. In some embodiments, the machine learning models may be configured to track the number of exercise activity repetitions.

In some other embodiments, the machine learning models may be configured to recognize or generate additional exercise activity types. The recognition or generation of additional exercise activity types may include detecting a user perform the “new” exercise activity for at least 5 sets of repetitions. For example, a user may begin a new sequence of exercise motions (e.g., “twisty-jump-spin-lunge”) and may want to track this sequence of physical activity. The machine learning models may generate custom motion filters for dynamically detecting and tracking such “new” exercise activity.

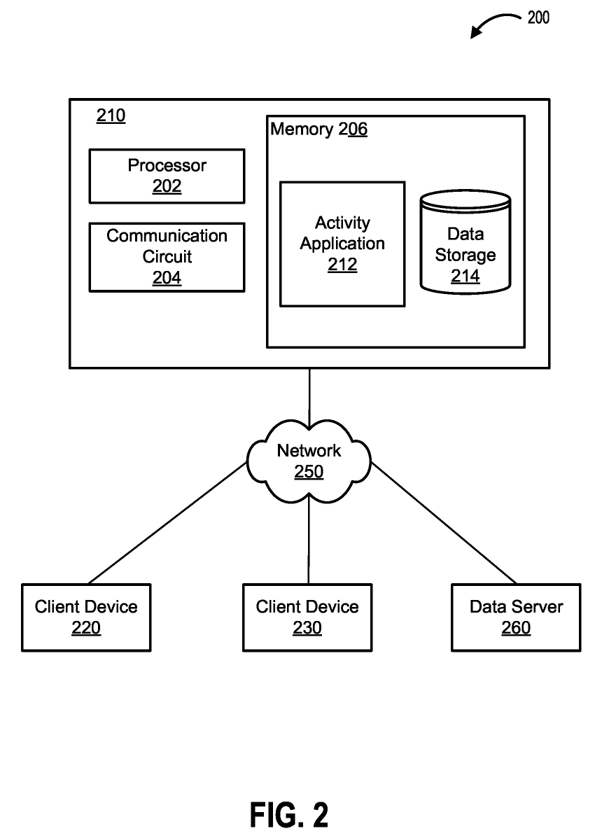

Reference is made to FIG. 2, which illustrates a block diagram of a fitness tracking platform 200, in accordance with embodiments of the present disclosure. The block diagram of the fitness tracking platform 200 may be an example of the fitness tracking platform 100 illustrated in FIG. 1.

A mobile computing device 210 may be configured to transmit or receive, via a network 250, data messages to or data messages from client devices (220, 230) or a data server 260. Two example client devices (220, 230) and a sole data server 260 are illustrated in FIG. 1. In some other examples, any number of client devices or subscription devices may be used.

To illustrate features of the fitness tracking system 200, the mobile computing device 210 may be a smart phone device. The smart phone device may be configured to communicate with client devices (220, 230) such as a smart watch device worn by a user or a pair of ear bud devices via the network 250. The smart phone device may be configured to communicate with the data server 260, such as a SaaS server or similar computing device, via the network 250.

In some embodiments, the mobile computing device 210 may communicate with the respective client devices (220, 230) or the data server 260 based on a common network communication protocol or based on different network communication protocols. For example, communication between the mobile computing device 210 and the client devices (220, 230) may be based on near-field communication protocols and the communication between the mobile computing device 210 and the data server 260 may be based on other wired or wireless network mediums.

The network 250 may include a wired or wireless wide area network (WAN), local area network (LAN), a combination thereof, or other networks for carrying telecommunication signals. In some embodiments, network communications may be based on HTTP post requests or TCP connections. Other network communication operations or protocols may be used.

In some embodiments, the network 250 may include near-field communication networks, such as Bluetooth™ networks, among other examples. In some examples, the network 250 may include the Internet, Ethernet, plain old telephone service line, public switch telephone network, integrated services digital network, digital subscriber line, coaxial cable, fiber optics, satellite, mobile, wireless, SS7 signaling network, fixed line, local area network, wide area network, or other networks, including one or more combination of the networks.

The mobile computing device 210 includes a processor 202 to implement processor-readable instructions that, when executed, configure the processor 202 to conduct operations described herein. The mobile computing device 210 may be configured to obtain data sets representing sensor data associated with physiological motion of a user and to dynamically generate predictions of user activity type or activity metrics in substantial real-time to the user. Other example operations will be described herein.

The processor 202 may be a microprocessor or a microcontroller, a digital signal processing processor, an integrated circuit, a field programmable gate array, a reconfigurable processor, or combinations thereof.

The mobile computing device 210 includes a communication circuit 204 configured to transmit or receive data messages to or from other computing devices, to access or connect to network resources, or to perform other computing applications by connecting to a network (or multiple networks) capable of carrying data. In some examples, the communication circuit 204 may include one or more busses, interconnects, wires, circuits, or other types of communication circuits. The communication circuit 204 may provide an interface for communicating data between components of a single device or circuit.

The mobile computing device 210 includes memory 206. The memory 206 may include one or a combination of computer memory, such as random-access memory, read-only memory, electro-optical memory, magneto-optical memory, erasable programmable read-only memory, and electrically-erasable programmable read-only memory, ferroelectric random-access memory, or the like. In some embodiments, the memory 206 may be storage media, such as hard disk drives, solid state drives, optical drives, or other types of memory.

The memory 206 may store an activity application 212 including processor-readable instructions for conducting operations described herein. In some examples, the activity application 212 may include operations for conducting machine learning operations associated with activity type prediction, operations associated with a recommendation application for providing exercise training recommendations in substantial real-time to a user during user exercise activity, or other example operations described in the present disclosure.

The mobile computing device 210 includes data storage 214. In some embodiments, the data storage 214 may be a secure data store. In some embodiments, the data storage 214 may store data sets received from the client devices (220, 230) or the data server 260. The data store 214 may be configured as a repository for data sets representing sensory data or other associated metadata from data-rich devices, such as smart watch devices, ear bud devices, smart garments, fitness tracker bands, among other devices (e.g., client devices 220, 230 or the data server 260). In some embodiments, the data store 214 may include a features store. A features store may be a local cache of user motion data representing motion data of one or more custom exercises. The features store may include one or more features sets which may be a raw data representation of exercise motion.

The client devices 220, 230 may be wearable computing devices such as smart watches, fitness tracking bands, smart eyewear, smart garments, wireless audio devices, among other examples. The wearable computing devices may be devices that a user may have adopted to wear routinely for one or more user exercise activities, such as while working out at a gym or exercising outdoors. The respective client devices 220, 230 may be configured as data-rich devices including sensors for detecting motion, patterns inherent in a sequence of motions, identifiable characteristics of detected motion, physical environment conditions, among other sensor-acquired data.

The respective client devices 220, 230 may include a processor, a memory, or a communication interface, similar to the example processor, memory, or communication interface of the mobile computing device 210. In some embodiments, the respective client devices 220, 230 may be computing devices associated with a local area network for transmitting or receiving signals to or from the mobile computing device 210. The local area network may include a wireless local area network or near-field communication networks such as Bluetooth™ or the like.

The data server 260 may be a computing device such as a data server, database device, or other data storing system for providing remote computing resources. For example, the data server 260 may conduct operations for managing or combining data sets from a plurality of mobile computing devices 210, where respective mobile computing devices 210 may conduct operations of the activity application 212.

In some embodiments, the data server 260 may be configured to provide gamification features or social media-related features to a plurality of users. For example, users of respective smartphone devices may opt to “follow” other users within a social network and compare exercise activity metrics with other users. In some examples, providing social-media related features can foster a community associated with exercise and healthy user lifestyles. In some embodiments, shared exercise activity metrics may be shared or kept private from other respective users.

In some embodiments, the data server 260 may provide gamification features to generate community competitions to incite friendly rivalry, and exercise activity level achievement rewards may be provided when users reach specific exercise activity level goals. In some embodiments, social media-related features may provide “leader boards” based on social groups associated with fitness centers attended, user profession, geographical location, age, or custom user groups. Social media-related features may motivate users to strive for and achieve fitness goals generated by the activity application 212 or created by respective users.

Example operations of the data server 260 described above may, in some embodiments, be conducted on the mobile computing device 210, or may be conducted on a combination of the data server 260 and the mobile computing device 210.

Embodiments of fitness tracking systems described herein may be configured to generate or obtain data sets representing sensor data from one or more data-rich devices (e.g., smartphone or wearable computing devices), dynamically track user exercise activity while the user may be at a gym, generate based on machine learning models predictions of specific user exercise activity type, and/or dynamically generate recommendations to the user during the user exercise activity. As such, embodiments of fitness tracking systems described herein may provide features of a virtual strength-training application for automatically identifying whether a user is doing squats or bench presses, push-ups or sit ups, or tally exercise repetitions. Further, the fitness tracking systems may be configured to generate user exercise activity metrics, such as rest time, range of motion, velocity, or the like, that may be transmitted to a live coach or trainer for progress monitoring.

To illustrate embodiments, the following examples illustrate a user who may be wearing or carrying at least one of a smart watch (e.g., Apple Watch™, or the like), wireless ear buds having one or more motion sensors therein (e.g., Apple AirPods™, or the like), or a smart phone (e.g., Apple iPhone™, Android-based smart phone, or the like) during an exercise or workout session. During a user’s exercise activity, the smart phone may conduct operations of an activity application 212 (FIG. 2) for obtaining substantially continuous, real-time data sets from the smart watch, wireless ear buds, or other user wearable devices for generating in substantial real-time predictions of the type of exercise activity that the user may be partaking in. The activity application 212 may provide one or more of the above-described generated predictions as feedback to the user via graphical user interfaces or audio interfaces.

In some embodiments, the activity application 212 may conduct operations to automatically detect the start of a workout activity and an end of the workout activity, without obtaining user input to indicate the start or conclusion of the workout activity. Upon detecting a start of a workout activity, the activity application 212 may be configured to dynamically generate a user interface for display at the smart watch or the smart phone. The user interface may be configured to provide a list of at least one predicted exercise associated with the machine learning model output, and the user may provide feedback on whether the predicted exercise activity predictions may be accurate. Such user feedback may be utilized for improving or training the machine learning model.

The activity application 212 may in substantial real-time determine one or a plurality of exercise activity statistics or details, such as range of user motion, velocity, acceleration, detected user rest time, physiological metrics of the user (e.g., heart rate, etc.) for providing the user with guidance or motivation through the exercise activity. Upon detecting a conclusion of the activity exercise or a repetition set, the activity application 212 may generate a summary of the user’s activity exercise. Data sets generated during user exercise activity may form the basis of training data sets for improving machine learning model output and may form the basis for providing future exercise activity guidance.

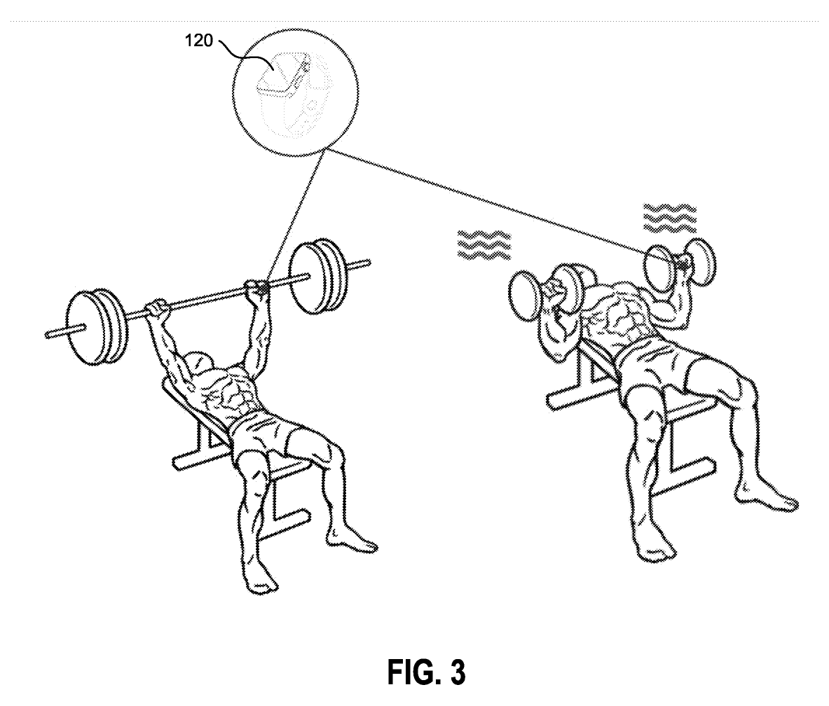

Reference is made to FIG. 3, which illustrates an example of a smart watch device 120 (FIG. 1) worn by a user partaking in weightlifting exercises, in accordance with embodiments of the present disclosure. The user may be wearing the smart watch device 120 on a wrist of the user.

In some embodiments, the smart watch device 120 may include one or more sensors configured to detect motion representing user movement or physical environment conditions. For example, the smart watch device 120 may include one or more of an accelerometer, a gyroscope, a magnetometer, or other sensors for detecting acceleration, gyroscopic motion, gravity, temperature, acoustic signals, atmospheric pressure, or magnetic field during exercise activity. Data sets associated with the detected motion may be for deriving or predicting the exercise activity type by the user.

FIG. 3 illustrates the user doing weightlifting exercises, such as bench presses with a barbell and, alternatively, with dumbbells. As the user may be wearing a smart watch device 120, the smart watch device 120 may generate a series of sensor data, and the series of sensor data may be used for generating predictions on the type of weightlifting exercise by the user.

Although both drawings in FIG. 3 show a user conducting bench press exercises, the respective drawings illustrate the user conducting bench press exercises based on different equipment. In some embodiments, the activity application 212 (FIG. 2) may conduct operations for distinguishing the type of activity / equipment used by the user based on characteristics derived from sequences of sensor data.

In one example, the user may be conducting bench press exercises with a barbell. In another example, the user may be conducting bench press exercises with dumbbells. The user’s wrist motion when conducting bench presses with a barbell may be different than the user’s wrist motion when conducting bench presses with dumbbells, at least because there may be greater variation in wrist movement when pushing up on dumbbells as compared to wrist movement when pushing up on a barbell.

In some situations, a user may be conducting one or more exercises associated with common physiological motion characteristics but may be different in user positioning. For example, a user partaking in bench press exercises with a barbell may exhibit upper body or arm motion, as detected by one or more sensors by a smart watch, similar to upper body or arm motion exhibited with the user partaking in overhead press exercises. However, the user partaking in bench press exercises may be lying down on a bench, whereas the user partaking in overhead press exercises may be in a partially upright, standing position. It may be beneficial to provide fitness tracking system features to combine data sets from two or more client devices to predict or infer an activity type with increased confidence levels / scores, thereby being able to increase exercise prediction accuracy to distinguish exercise activities having common physiological motion characteristics, but that may nonetheless be different exercise activities.

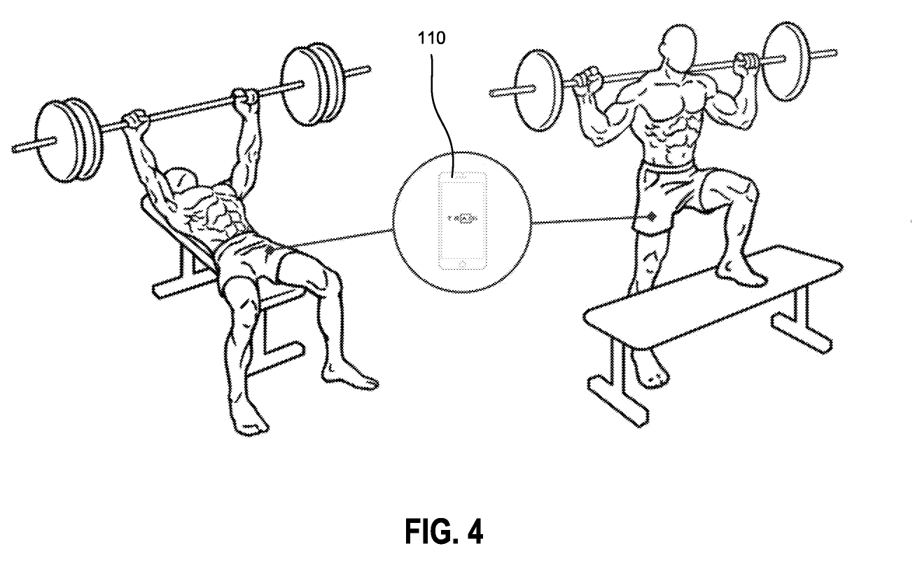

Reference is made to FIG. 4, which illustrates the mobile computing device 110 (FIG. 1) carried by the user in a garment pocket, in accordance with embodiments of the present disclosure. In FIG. 4, the user may also be wearing a smart watch device (not explicitly illustrated in FIG. 4).

The mobile computing device 110 may be in communication with the smart watch device, and may obtain substantially continuous, real-time data sets from the smart watch device representing physiological motions of the user’s wrist / arm movement.

The drawings in FIG. 4 illustrate the user partaking in bench press exercises and the user, subsequently, partaking in standing press exercises. The mobile computing device 110 may conduct operations of the fitness application 112 (FIG. 1) for predicting that the user is partaking in one of either bench press exercises or standing press exercises. In the present example, the motion detected by the smart watch device when the user partakes in bench press exercises or the standing press exercises may be similar. The mobile computing device 110 may generate a prediction on the type of exercise being conducted and may display the predictions on a user interface for the user to confirmation input on.

To increase confidence levels / scores associated with predicting the exercise activity by the user, the computing device 110 may in some embodiments generate predictions based on data sets from two or more computing devices. In the example illustrated in FIG. 4, the orientation of the mobile computing device 110 in three dimensional space may be different when: (i) the user is lying on a bench when partaking bench press exercises; and (ii) the user is in a substantially standing position when partaking in standing overhead press exercises.

Thus, in some embodiments, the mobile computing device 110 may predict the exercise activity type of the user based on a combination of sensor data sets from the smart watch device and based on orientation data sets associated with the mobile computing device 110. For example, when the mobile computing device 110 is in an upstanding position relative to the earth, the user is less likely to be performing bench press exercises when upper body / arm movements are detected. Further, when the mobile computing device 110 is in a position substantially parallel to the earth (e.g., when the user is lying down on a bench with the mobile computing device 110 is in the user’s garment pocket), the user is less likely to be performing standing overhead press exercises. Thus, embodiments of the fitness tracking system described herein may be configured to generate predictions associated with user motion as detected by one or a combination of client devices (e.g., smart watch devices, smart garments, etc.) and to track user motion for generating a series of exercise activity records.

In some embodiments, the mobile computing device 110 may aggregate or combine the series of exercise activity records for storage at a data storage or for transmission to a remote / off-site data server 160. Aggregation of data sets from data-rich computing devices may be the basis for predicting exercise activity based on a plurality of data sets associated with users across user body types, geographies, profiles, or the like. Data sets associated with exercise activities of a pool of users may be used for predicting exercise activities of individual users. Machine learning models of the activity application 212 (FIG. 2) may be iteratively trained and dynamically re-trained for improving exercise activity predictions.

Embodiments of the activity application 212 (FIG. 2) may include operations for detecting type of equipment that a user may be partaking in. As an example, referring again to FIG. 3, the user may be partaking in bench press exercises. In one drawing, the user may be conducting bench presses with a barbell. In another drawing, the user may be conducting bench presses with dumbbells.

It may be beneficial to provide methods of increasing confidence scores / levels of exercise activity predictions based on detection of user motion associated with pre-activity or post-activity. For example, the user may be setting up for conducting bench presses with a barbell, the user may place disc weights at opposing sides of the barbell. The mobile computing device (not explicitly illustrated in FIG. 3) may conduct operations for detecting motion characteristic of a user placing disc weights on opposing sides of the barbell (via sensors on the smart watch device and data sets transmitted to the mobile computing device), such that these detected motion characteristics may be combined with data sets obtained during the actual exercise activity for predicting that the user may be partaking in bench presses with a barbell.

Further, when the user may be handling a barbell for bench press exercises, the mobile computing device may detect that the user motion may suggest the equipment substantially moving along a single axis (e.g., vertically relative to the earth), and may predict that a barbell is being used for exercises.

In contrast, when the user may be setting up for conducting bench presses with dumbbells, the user may pick up respective dumbbells and may exhibit wrist rotation motion to setup the dumbbells in the desired position for the bench press operations. For example, the mobile computing device 110 may conduct operations to identify that equipment being handled based on user motion is about multiple axis, thereby suggesting that dumbbells may be used by the user.

Accordingly, the mobile computing device (not explicitly illustrated in FIG. 3) may conduct operations for detecting motion characteristics of a user rotating dumbbells into a desirable position for bench press exercises, such that these detected motion characteristics may be combined with data sets obtained during the actual exercise activity for predicting that the user may be partaking in bench presses with dumbbells.

In some embodiments, mobile computing devices may be configured to provide at a user interface recommendations for exercise activity based on an associated user’s profile, based on the user’s prior exercise activity logs, or based on externally determined user data. In some embodiments, externally determined user data may include data sets representing user stress levels over time, user sleep quality or sleep patterns, user’s log of recent diet, or user’s log of other physiological data (e.g., any menstrual cycle data, medication usage data, among other examples). Exercise activity recommendations may be based on holistic data associated with the user’s well-being, such as the user’s sleep cycle patterns, records of whether the user is eating healthy meals based on predefined nutrition guidelines. In some embodiments, externally determined data sets may include data associated with historical patterns of the user’s workout routine (e.g., working out leg exercises every Monday, etc.).

In some embodiments, externally determined user data may be obtained based on interfaces with other applications executed on the mobile computing device. For example, the mobile computing device may obtain a user’s menstrual cycle from third-party applications such as Flo, or may obtain a user’s sleep cycle patterns, diet records, heart rate data or blood pressure data from third-party applications or from applications that may be native to the Apple iOS™ environment. In some embodiments, externally determined user data may include the user’s sleep cycle patterns, diet records, heart rate data or blood pressure data from third-party applications or from applications that may be native to the Android™ environment or other operating system environments.

Based on user data obtained from third party applications, the mobile computing device may be configured to provide recommendations to alter or tweak the user’s daily lifestyle in combination with the user’s exercise activity plans.

In some embodiments, the mobile computing devices may be configured to provide a post-workout analysis for providing workout results, including total volume lifted, average health metrics, among other examples. The post-workout feedback may include recommended future workout routines, followed by recommended diet plans or recovery times.

In some embodiments, the mobile computing devices may be configured to determine whether a user may reach an exercise activity plateau. An exercise activity plateau may be identified when the user may reach a point of muscle fatigue in their workout, and the user may be no longer able to exercise that muscle group effectively. In some embodiments, machine learning models may be trained to provide recommendations on max weights for repetitions and for best potential weights (e.g., dumbbells) to utilize for maximizing the user’s workout potential.

Referring again to FIG. 1, the fitness tracking platform 100 may include one or more wearable computing devices, such as a smartwatch device 120, an audio device 130, or other wearable computing devices. In some embodiments, the fitness tracking system 100 may be configured to combine data sets from two or more client devices, such as the smartwatch device 120 and the audio device 130, among other wearable devices, to predict an activity type with increased confidence or precision. Such example fitness tracking platforms may be configured to generate exercise activity predictions with increasing confidence or precision.

It may be beneficial to provide a fitness tracking platform configured to generate exercise activity predictions, exercise activity repetition counts, feedback representing exercise form evaluation, among other types of user feedback outputs with increasing confidence or accuracy based on operations of substantially one wearable computing device, such as a smart watch. That is, in some situations, a user may be performing exercises while donning a primary wearable computing device, while leaving other computing devices (e.g., mobile phone, audio headsets, etc.) at other physical locations such that the primary wearable computing device may not be in communication with these other computing devices.

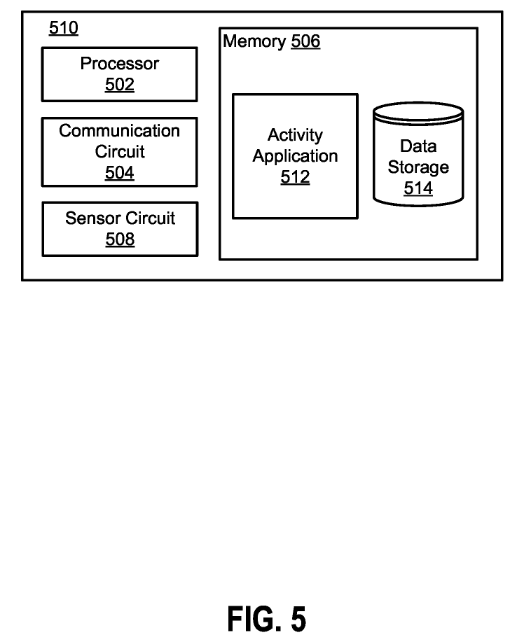

Reference is made to FIG. 5, which illustrates a block diagram of a wearable computing device 510, in accordance with embodiments of the present disclosure. The block diagram of the wearable computing device 510 may be an example smart watch, such as an Apple Watch™, Android™-based smart watch, fitness tracking bands, smart eyewear, smart garments, wireless audio devices, or other type of wearable computing devices. The wearable computing device 510 may be adopted to be worn or donned by a user during one or more exercise activities, such as while working out at a gym or exercising outdoors. The wearable computing device 510 may be configured as a data-rich device, including sensors for detecting motion, patterns inherent in a sequence of motions, identifiable characteristics of detected motion, physical environment conditions, among other sensor-acquired data.

The wearable computing device 510 may include a processor 502, such as a microprocessor or a microcontroller, a digital signal processing processor, an integrated circuit, a field programmable gate array, a reconfigurable processor, or combinations thereof.

The wearable computing device 510 may include a communication circuit 504 configured to transmit or receive data messages to or from other computing devices, to access or connect to network resources, or to perform other computing applications by connecting to a network (or multiple networks) capable of carrying data. The communication circuit 504 may be similar to the communication circuit 204 described with reference to FIG. 2.

The wearable computing device 510 may include memory 506. The memory 506 may store an activity application 512 including processor-readable instructions for conducting one or more operations described herein, such as for conducting machine learning operations associated with exercise type prediction, operations for providing exercise training recommendations in substantial real-time to a user during user exercise activity, operations for evaluating user exercise from, or operations for providing exercise training recommendations in substantial real-time to a user during an exercise activity.

The wearable computing device 510 may include a data storage 514. The data storage 514 may be a secure data storage and may store data sets generated by one or more sensor circuits 508.

The one or more sensor circuits 508 may include one or more accelerometers, gyroscopes, pedometers, magnetometers, or barometers, among other examples. The sensor circuit 608 may be configured to generate data sets representing movement or environmental conditions associated with the wearable computing device 510, such as tilt, shake, rotation, acceleration, or swing, among other examples. As will be described, based on one or more identified user movements or physical environment conditions, the wearable computing device 510 may be configured to predict or infer a type of exercise activity being undertaken by a user.

In some embodiments, the wearable computing device 510 may be configured to predict or infer a type of exercise activity in substantial real-time for providing feedback to the user. As an example, a user donning the wearable computing device 510 may conduct pre-exercise activity, such as approaching a dumbbell, lifting the dumbbell, and beginning several repetitions of bicep curls with the dumbbell. Based on sensor data sets generated by the sensor circuit 508, the wearable computing device 510 may be configured to identify the exercise activity prediction (e.g., bicep curl exercise) within several hundred milliseconds, and provide the exercise activity prediction at an output interface within 1 or 2 seconds. Other example time ranges for generating exercise activity predictions and providing the exercise activity prediction at an output interface may be contemplated.

In some embodiments, a series of sensor data generated by a wearable computing device may be configured to generate an exercise prediction based on detected movement of the wearable computing device. For example, when a user wears a smart watch (e.g., Apple Watch™) on their wrist and engages in one or more weightlifting or other conditioning exercises at a fitness gym, the smart watch may be configured to generate a prediction of the exercise type undertaken by the user. For example, the wearable computing device may be configured to generate predictions that a user is conducting bicep curls, bench presses, shoulder presses, among other example exercises.

In some examples, a given exercise may be performed using two or more different types of equipment. For example, bench press exercises may be performed using dumbbells, barbells, or a Smith machine. In another example, bicep curls may be performed using dumbbells or barbells. In another example, shoulder presses may be performed using barbells or a shoulder press machine. It may be desirable to provide fitness tracking devices for generating exercise predictions with greater granularity or precision based on sensor data associated with motion of a user’s limb.

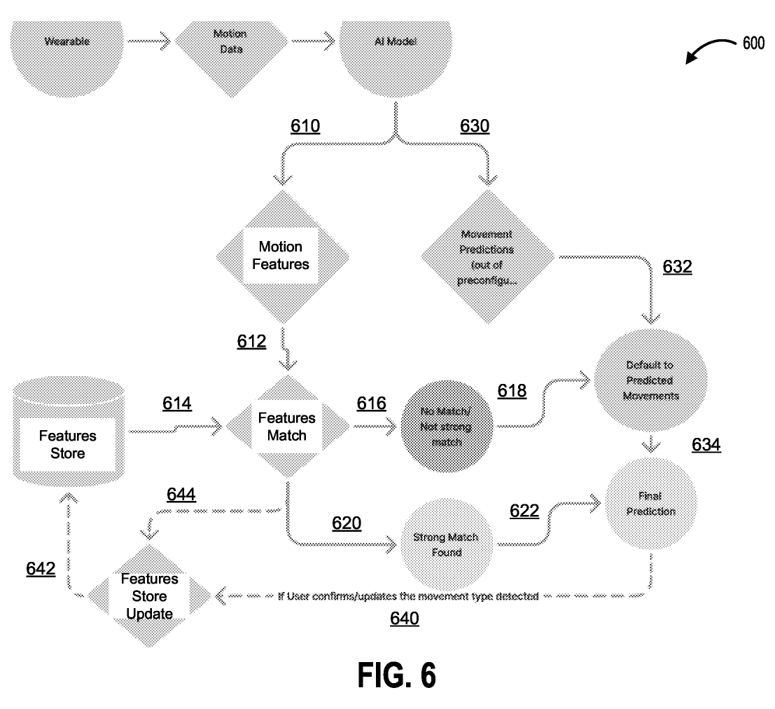

Reference is made to FIG. 6, which illustrates a flowchart of an exercise prediction method 700, in accordance with embodiments of the present disclosure. The method 600 may be conducted by the mobile computing device 110 of FIG. 1 (or the mobile computing device 210 of FIG. 1). The processor-executable instructions may be stored in memory and may be associated with the activity application 212 (FIG. 2) or other processor-executable applications not illustrated in FIG. 2. The method 600 may include operations such as data retrievals, data manipulations, data storage, or other operations, and may include computer-executable operations.

The mobile computing device 110 may receive data messages from one or more client devices. For example, the one or more client devices may be wearable computing devices having sensors thereon for detecting motion of the user.

At operation 602, the mobile computing device 110 may receive raw motion data from a client device, such as a wearable computing device. The motion data may be generated based on one or more of gyroscope devices, accelerometer devices, magnetometer devices, among other example devices. The raw motion data may be generated based on a smart watch device donned on the user’s wrist. In some examples, the raw motion data may be generated based on other wearable devices, such as ear-bud devices for audio, among other examples.

In some embodiments, the raw motion data may include time series data representing movement characteristics such as tilting, shaking, rotation, acceleration, or swinging of the client device over time. The raw motion data may be a proxy representing user movement during exercise activity.

In some embodiments, the raw motion data may include sensor data for representing environmental conditions. For example, the raw motion data may include magnetic field strength or magnetic field direction data associated with equipment that may be nearby the user’s limb. For example, magnetometer sensor data may be for inferring whether a user may be grasping a dumbbell or other equipment.

At operation 604, the mobile computing device 110 may conduct operations for cleaning, filtering, or otherwise transforming the raw motion data to provide a sensor data set for downstream prediction operations. In some embodiments, operations for cleaning, filtering, or transforming the raw motion data may include operations for aligning time stamped data or for identifying and discarding data representing noise, among other examples.

At operation 630, the mobile computing device 110 may generate an exercise prediction based on a prediction model and the sensor data set. In some embodiments, the prediction model may be defined by one or more oscillating signal profiles representing characteristic signal profiles of expected sensor data while a user’s limb may be conducting an exercise.

Upon identifying that the sensor data set is representative of a particular set of motions for an exercise, the mobile computing device 110 at operation 632 may determine that the sensor data set is representative of a predicted exercise prior identified during training of the prediction model.

In some scenarios, the prediction model may be unable generate an exercise prediction with high confidence. For example, the sensor data set may represent motion that does not substantially mirror an oscillating signal profile of a prior-identified exercise activity and the mobile computing device 110 may be unable to generate an exercise prediction with a desired level of confidence. It may be desirable to provide systems and methods for update or train the prediction model based on newly identified series of movements.

At operation 610, the mobile computing device 110 may transform the sensor data set to motion features. In some embodiments, motion features may be data structures representing motion data generated based on movement of wearable computing devices.

At operation 612, the mobile computing device 110 may conduct comparison operations comparing motion features representing movement of a wearable computing device over time with template motion features stored in a features store. For example, the mobile computing device 110 may retrieve (at operation 614) template motion features representing known exercise movement at a user’s wearable device and may conduct comparison operations for comparing motion features representing raw motion data (from operation 602) and template motion features representing prior-known exercise movement.

In a scenario where the motion features representing raw motion data from operation 602 substantially corresponds to at least one template motion feature from the features store, the mobile computing device 110, at operation 620, will identify the substantially matching oscillating signal profile.

At operation 622, the mobile computing device 110 may generate the exercise prediction based on the comparative operations of motion features.

In a scenario where the motion features representing raw motion data from operation 602 do not substantially correspond to any template motion features available at the features store, the mobile computing device 110 at operation 616 may generate a signal indicating that there is no template feature representing an oscillating signal profile of any exercise activity that substantially corresponds to the motion feature representing raw motion data from operation 602.

At operation 618, the mobile computing device 110 may generate the exercise prediction based on the prediction model and the sensor data set (akin to operation 630). In some embodiments, the prediction model may be based on machine learning operations based on one or a combination of TensorFlow™ library operations, CoreML™ library operations, or Pytorch library operations. In some embodiments, the prediction model may be trained and defined by one or a series of oscillating signal profiles associated with one or more user limb movement stages for an associated exercise.

At operation 618, the mobile computing device 110 may generate the exercise prediction based on a nearest-identified exercise prediction. In some examples, a nearest-identified exercise prediction may represent an exercise having a motion signal profile most similar to the motion represented by the raw motion data retrieved from operation 602.

As an example, a nearest-identified exercise prediction may indicate that the exercise is a bench press exercise when the raw motion data may be associated with another exercise that has similar characteristics to a bench press exercise but does not substantially align with motion characteristics of the bench press exercise.

At operation 634, the mobile computing device 110 may generate a signal for displaying the exercise prediction at a display to the user. The signal for displaying the exercise prediction may be for generating a user interface soliciting feedback from the user on whether the exercise prediction is representative of the exercise activity undertaken by the user.

In a scenario where the motion features representing raw motion data from operation 602 substantially corresponds to a template motion feature available at the features store, at operation 640, the mobile computing device 110 may receive a signal representing an indication from the user that the exercise prediction is representative of the exercise activity undertaken by the user. That is, the signal representing the indication from the user may be user validation that the exercise prediction is accurate.

In some embodiments, where the signal represents an indication that the exercise prediction is representative of the exercise activity, the mobile computing device 110 may conduct operations to generate a features store update to refine template motion features. Refining template motion features may represent operations for training the prediction model and for refining operations for generating exercise predictions.

In a scenario where the motion features representing raw motion data from operation 602 do not substantially correspond to any template motion features available at the features store, the mobile computing device 110 at operation 640 may receive a signal that the exercise prediction is not representative of the exercise activity undertaken by the user. That is, the signal may represent user feedback that the exercise prediction is not accurate.

Where the mobile computing device 110 generates refined template motion features, at operation 642, the mobile computing device 110 may update the features store with the refined template motion features. Iteratively updating the features store with refined or additionally generated template motion features continually refines the prediction model and system for predicting user exercises.

In some embodiments, operations for generating refined template motion features for storing at the features store may be based on one of a plurality of scenarios or inputs received by the mobile computing device 110 at operation 640.

In a first scenario, the mobile computing device 110 may receive a signal indicating that the exercise prediction is representative of the exercise activity undertaken by the user. The exercise prediction may be based on a mapping to a substantially similar motion feature already stored in the features store or based on an exercise prediction based on the prediction model. In the present scenario, the mobile computing device 110 may generate a signal to confirm or refine a template motion feature previously stored at the features store. Such operations may bolster or strengthen confidence in generating exercise predictions.

In a second scenario, the mobile computing device 110 may receive a signal at operation 640 that the exercise prediction is not representative of exercise activity undertaken by the user. The exercise prediction may be based on a nearest-identified exercise prediction. In a scenario where the exercise prediction is based on a nearest-identified exercise prediction, the mobile computing device 110 (at operation 740) may receive the signal that (i) the exercise prediction is not representative of the exercise activity undertaken; and (ii) an alternate exercise prediction may be better representative of the exercise activity taken.

As an example, the mobile computing device 110 may have generated a signal for displaying two or more exercise predictions at a display ranked based on increasing prediction confidence. In a scenario where the mobile computing device 110 receives user feedback that indicates an exercise prediction associated with a lower confidence is more representative (e.g., a “second most” confident prediction), the mobile computing device 110 may generate a motion feature based on the user feedback for storing at the features store in association with the sensor data set identified at operation 604.

In a third scenario, the mobile computing device 110 may receive a signal at operation 640 that none of the displayed list of exercise predictions can be representative of the exercise activity undertaken. In some embodiments, the list of exercise predictions may be an ordered list based on prediction confidence level. In the present scenario, the mobile computing device 110 may not have previously been trained to identify the exercise activity associated with the sensor data set generated at operation 604. It may be desirable to generate trained features or to train the prediction model for the previously unidentified exercise activity.

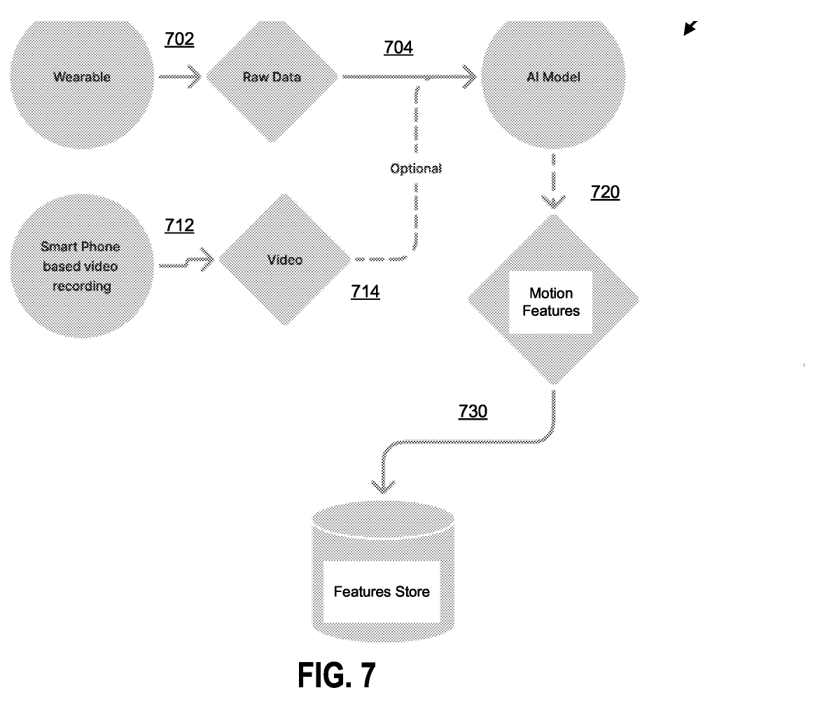

Reference is made to FIG. 7, which illustrates a flowchart of an exercise prediction method 700, in accordance with embodiments of the present disclosure. The method 700 may be conducted by the mobile computing device 110 of FIG. 1 (or the mobile computing device 210 of FIG. 1). The processor-executable instructions may be stored in memory and may be associated with the activity application 212 (FIG. 2) or other processor-executable applications not illustrated in FIG. 2. The method 700 may include operations such as data retrievals, data manipulations, data storage, or other operations, and may include computer-executable operations.

At operation 702, the mobile computing device 110 may receive raw motion data from a client device, such as a wearable computing device. The motion data may be generated based on one or more of gyroscope devices, accelerometer devices, magnetometer devices, among other example devices. The raw motion data may be generated based on a smart watch device donned on the user’s wrist.

Similar to embodiments described with reference to FIG. 6, the raw motion data may include time series data representing movement characteristics such as tilting, shaking, rotation, acceleration, or swinging of the client device over time. The raw motion data may be a proxy representing user movement during exercise activity.

In a scenario described with reference to operation 640 (FIG. 6) where the mobile computing device 110 receives a signal representing that none of the displayed list of exercise predictions are representative of the exercise activity undertaken, the mobile computing device 110 may be configured to generate additional template motion features to be associated with exercise activity. In some embodiments, generation of additional template motion features may be based on a combination of raw motion data and video recordings associated with movement of the raw motion data.

At operation 712, the mobile computing device 110 may obtain a video data set associated with the user conducting exercise activities. The video data set may be associated with video footage of the user partaking in the exercise activity.

In some embodiments, the mobile computing device 110 may include one or more image capture devices for generating the video data set. For example, the exercise user may setup the mobile computing device 110 for recoding video whilst the user is conducting the exercise, and the mobile computing device 110 may generate the video data set for representing the conducted exercise activity.

At operation 714, the mobile computing device 110 may transform the video data set to a data structure suitable for combining with raw motion data obtained at operation 702.

In some embodiments, a combination of video data set and raw motion data set may be used for determining when an exercise activity started and when an exercise activity ceased. In some embodiments, the video data set may be used for determining an exercise activity type and for validating (manual or automated) the exercise activity type.

At operation 704, the mobile computing device 110 may align the obtained raw motion data (at operation 702) with the video data set (obtained at operation 714) to generate a time-series data set having a combination of motion data temporally aligned with video data representing the user conducting the exercise activity.

At operation 720, the mobile computing device 110 may generate one or more motion features based on a features model and the time-series data set having the combination of motion data temporally aligned with video data representing the user conducting the exercise activity. In some embodiments, the features model may be based on machine learning operations based on one or a combination of TensorFlow™ library operations or CoreML™ library operations. The generated motion features are associated with the newly identified exercise motion or activity and may be template features for downstream operations for predicting user exercise motion or user exercise activity. In some embodiments, a features model may be configured as an LSTM+ Transformer based neural network for processing chunks of raw motion data associated with periodic movements. In some embodiments, the features model may be configured based on other machine learning architectures.

At operation 730, the mobile computing device 110 may update the features store with the generated motion features for downstream operations to predict user exercise motion or activity. The generated motion features may be subsequently iteratively updated to be refined to support user exercise prediction. In some embodiments, the generated motion features may represent or be associated with a user-customized exercise prediction model for future exercise predictions associated with that user. The user-customized exercise prediction model may supplement the prediction model described with reference to FIG. 6.

In some scenarios, the prediction model described with reference to FIG. 6 may be a model representing a set of common motion features trained based on a gaussian distribution of exercise users. The user-customed prediction model described with reference to FIG. 7 may supplement the prediction model described with reference to FIG. 6 and may represent refinements for specific users.

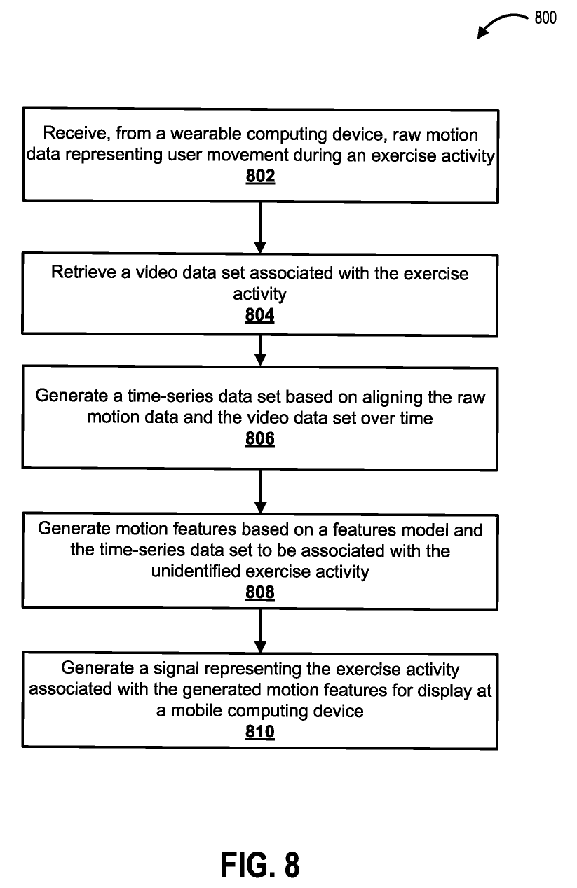

Reference is made to FIG. 8 which illustrates a flowchart of a method 800 of fitness exercise tracking, in accordance with embodiments of the present disclosure. The method 800 illustrated in FIG. 8 may include operations conducted by one or a combination of a wearable computing device worn on a user limb or by the mobile computing device 110 (FIG. 1). For example, a wearable computing device may be a smart watch device or a fitness tracking band configured to be donned on a user’s wrist. The wearable computing device may be the wearable computing device 510 of FIG. 5.

The method 800 may include operations conducted by one or more processors of the wearable computing device or the mobile computing device and may include operations such as data retrievals, data manipulations, data storage, or other operations, and may include computer-executable operations.

In some embodiments, the wearable computing device configured to be worn on a user limb, such as a user’s wrist, may include a sensor circuit configured to generate sensor data. The sensor circuit may include one or more of accelerometers, gyroscopes, pedometers, magnetometers, or barometers, among examples of sensor devices.

As described, the wearable computing device may include one or more sensors for detecting movement or other environmental conditions, and may generate a sequence or series of sensor data over time (e.g., time-series sensor data set). The fitness tracking device may store the sensor data for generating exercise predictions, determining exercise activity repetition counts, determining exercise form quality, generate exercise recommendation routines, among other signals, for providing feedback to a user in substantial real-time.

At operation 802, the mobile computing device may receive, from the wearable computing device, raw motion data representing user movement during an exercise activity.

At operation 804, the mobile computing device may retrieve a video data set associated with the exercise activity. In some embodiments, the mobile computing device may be a smartphone device including a video or image capture device.

In a scenario where an exercise activity may not have been previously identified or predicted, the mobile computing device may be set up for capturing video data of a user performing an exercise activity. Such video data of the user preforming the newly identified exercise activity may be combined with raw motion data captured concurrently with the video data for generating motion features for downstream exercise activity predictions.

At operation 806, the mobile computing device may generate a time-series data set based on aligning the raw motion data and the video data set over time.

At operation 808, the mobile computing device may generate motion features based on a features model and the time-series data set to be associated with the unidentified exercise activity.

At operation 810, the mobile computing device may generate a signal representing the exercise activity associated with the generated motion features for display at the mobile computing device.

The artificial intelligence model implemented by described embodiments of fitness tracking systems herein can include any suitable type of AI model or combination of models capable of processing motion data and generating exercise-related insights. Examples of AI models that may be used include one or a combination of convolutional neural networks (CNNs), recurrent neural networks (RNNs), long short-term memory networks (LSTMs), transformers, decision trees, support vector machines (SVMs), and ensemble models. The model may be trained to recognize patterns in motion data and correlate those patterns with specific exercises, repetition counts, movement quality indicators, and effort metrics. The AI model can be executed entirely on a single computing device, such as a server or a mobile computing device (e.g., a smart phone), or its computational workload can be distributed across multiple devices. For example, portions of the model may be run on a server, a mobile computing device, and one or more wearable computing devices (e.g., a smart watch), enabling efficient real-time processing and responsiveness while leveraging the capabilities of each device in the system.

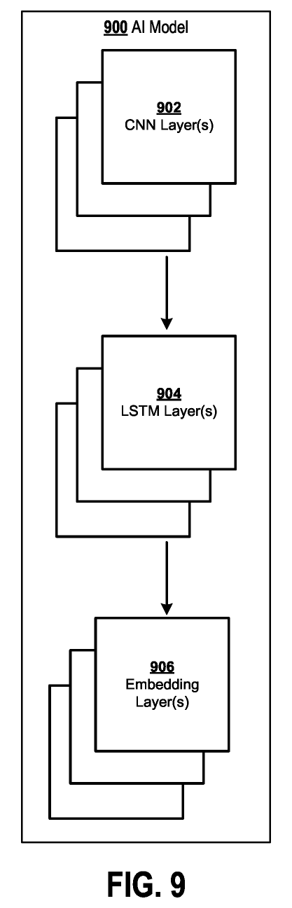

A diagram of an example embodiment of an AI model 900 that can be implemented by the described fitness tracking systems is provided in FIG. 9. As shown, the AI model 900 can include one or more convolutional neural network (CNN) layers 902, one or more long short-term memory (LSTM) layers 904, and one or more embedding layers 906. Each of these layers is described in further detail below.

The CNN layer(s) 902 can include any suitable number or combination of CNNs. Each CNN can be a deep learning model configured to scan data provided as input to the CNN with convolutional filters that detect local patterns in the data. The CNN layer(s) 902 can allow the AI model 900 to learn and generalize complex patterns in raw motion data provided by sensors in a wearable computing device (e.g., a smart watch) and thereby enable the AI model 900 to identify spatial and temporal correlations in the raw motion data.

In some embodiments, the CNN layer(s) 902 can be configured to receive data characterizing raw motion of a user (e.g., accelerometer or gyroscope data provided by one or more sensors of a wearable computing device) as input. The CNN layer(s) 902 can transform this raw motion data to identify motion features, assign weights to the identified features based on learned parameters, and store those features for further processing by downstream components of the model. This architecture can enable efficient and scalable analysis of motion data and can support accurate and real-time recognition of exercise movements.

In some embodiments, the LSTM layer(s) 904 can be downstream of the CNN layer(s) 902 and can include any suitable number or combination of LSTM networks. Each LSTM network can be a recurrent neural network (RNN) configured to learn and retain information across time steps. Each LSTM layer 904 can incorporate memory cells and gating mechanisms that enable the AI model 900 to selectively retain or discard information, allowing it to capture long-range dependencies and temporal patterns in motion data. In some embodiments, the LSTM layer(s) 904 can be configured to receive motion features extracted by preceding layers (e.g., the CNN layer(s) 902) and analyze how those features evolve over time. This temporal analysis can support accurate identification of exercise repetitions, assessment of movement consistency, and evaluation of effort dynamics throughout a workout session.

The embedding layer(s) 906 can be downstream of the LSTM layer(s) 904 and can include any suitable number or combination of embedding models. Each embedding layer can be configured to transform categorical or high-dimensional input data into dense, lower-dimensional vector representations. The embedding layer(s) 906 can enable the AI model 900 to efficiently encode contextual information, such as user-specific physiological attributes (e.g., height, weight, gender), exercise types, or device-specific sensor characteristics. These embeddings can allow the model 900 to incorporate personalized context into its analysis, improving its ability to generalize across users while maintaining sensitivity to individual differences.

The embodiments of the AI model 900 are presented for illustrative purposes and is not intended to be limiting. An AI model implemented by a fitness tracking system can include a different combination of model types than the combination illustrated in FIG. 9, and can be configured with any number of layers, nodes, weights, biases, activation functions, and other parameters. The architecture of an AI model can be selected or adapted based on the nature of the motion data being processed, the desired output metrics, and the computational resources available, allowing for flexible and scalable implementation across a variety of fitness tracking scenarios.

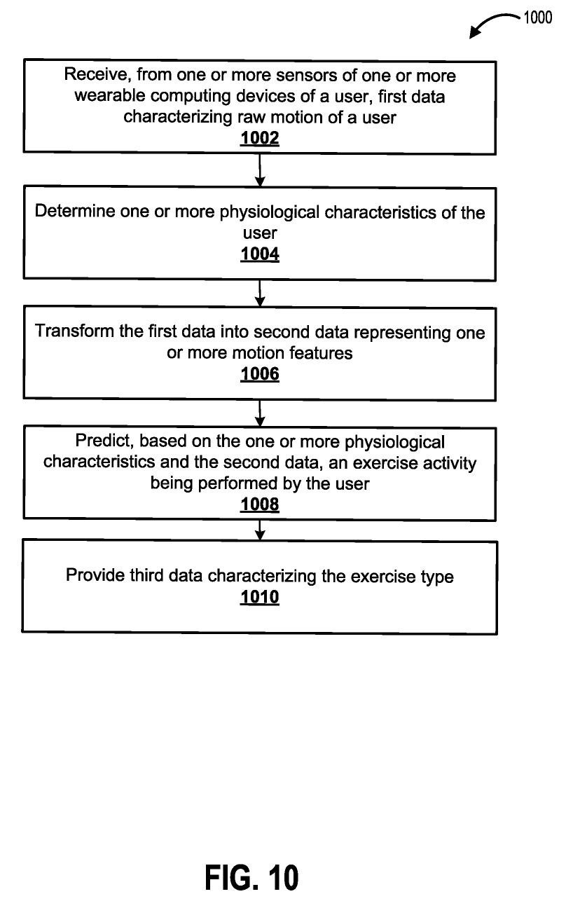

FIG. 10 illustrates another fitness tracking method 1000, in accordance with embodiments of the present disclosure. The method 1000 can be performed, all or in part, by one or more processors of components of a fitness tracking system, for example, processor(s) of a mobile computing device (e.g., a smart phone), a wearable computing device (e.g., a smart watch), and/or other computing devices (e.g., a server) that constitute a fitness tracking system. In some embodiments, the method 1000 can be implemented as instructions stored in non-transitory memory. Alternatively, or in addition, the method 1000 can be included in non- transitory computer readable memory storing the method 1000 as instructions which, when executed by one or more processors forming part of a fitness tracking system, causes the processor(s) to perform operations of the method 1000.

At 1002, first data characterizing raw motion of the user is received from one or more sensors of one or more wearable computing devices of a user. The wearable computing device(s) can include a smart watch, a fitness tracking band, wireless headphones or earphones, and/or the like. The sensor(s) of the wearable computing device(s) can include accelerometers, gyroscopes, magnetometers, or other motion-sensing components capable of capturing physical movement. The first data can be received by another computing device, for example, a mobile computing device (e.g., a smart phone), a personal computer, or a server that is communicatively coupled to the wearable computing device(s). In some embodiments, the first data can be transmitted from the wearable computing device(s), for example, over a wireless communication network (e.g., a Bluetooth connection), with minimal latency (e.g., less than one second from measurement), enabling timely and responsive analysis of user activity.

In some embodiments, the first data includes a time series of vectors representing how the user (or a specific body part of the user, such as the user’s wrist or head) is accelerating in three-dimensional space. Each vector in the time series can encode both the magnitude and direction of acceleration at a given time step, providing a detailed representation of the user’s motion over time. The size of the first data set may vary depending on the sampling frequency of the sensors, which may range from tens to hundreds of measurements per second.

In some embodiments, the first data can include data characterize user motion during a exercise activity as well as during periods of time preceding and following the exercise activity. For example, the first data can include motion data for approximately five seconds (or another duration) before and five seconds (or another duration) after an exercise is performed to provide additional context for exercise detection. This pre- and post-exercise motion data can indicate movements such as placing weights on a barbell, picking up dumbbells, walking to or from a squat rack, sitting down or lying on a bench, or other preparatory or concluding actions. Although these movements are not part of the exercise itself, they can provide valuable clues that can improve the accuracy of exercise identification by indicating the type of equipment being used and the user’s intended activity.

Upon receipt at 1002, the first data can be provided as input to an AI model implemented by the fitness tracking system (e.g., the AI model 900 described with respect to FIG. 9). In some embodiments, the first data can be provided to the AI model without undergoing any preprocessing. This can enable the model to learn and extract relevant features from raw motion signals and thereby preserve the full fidelity and variability of the data, which can improve generalization and adaptability across diverse users and movement styles. In other embodiments, the first data can be preprocessed (e.g., filtered, smoothed, normalized, etc.) prior to being provided as input to the AI model. Preprocessing the first data can reduce noise, enhance signal clarity, and improve model stability and performance by presenting cleaner, more consistent inputs for feature extraction and prediction.

At 1004, one or more physiological characteristics of the user are determined. In some embodiments, these characteristics can be identified using the AI model implemented by the fitness tracking system. The AI model can utilize information learned during previous fitness tracking sessions to infer physiological characteristics based on historical motion data and user behavior. In some embodiments, data regarding physiological characteristics of the user can be stored by one or more computing devices of the fitness tracking system, such as the wearable computing device or a mobile computing device, and accessed during analysis. Additionally, or alternatively, data regarding the user’s physiological characteristics may be provided directly by the user through manual input.

The determined physiological characteristics can be any characteristics that influence how the user moves while exercising. Examples of such physiological characteristics include (but are not limited to) the user's height, weight, age, gender, limb length, body composition, and overall fitness level, all of which can affect the range of motion, acceleration profiles, and movement dynamics of the user.

In addition to determining static physiological characteristics, in some embodiments, a current physiological state of the user can be assessed. Assessing the current physiological state of the user can involve determining (e.g., by the AI model and/or by accessing data stored by one or more computing devices of the fitness tracking system) information such as the amount of sleep or rest the user has had over a recent period (e.g., the preceding 24 hours), whether the user is menstruating, the user's hydration level, the user’s nutritional intake (e.g., the amount of protein, carbohydrates, fats, or other nutrients consumed recently), and/or the like. Other examples of physiological state data may include stress levels, recent illness or injury, medication usage, and environmental factors such as ambient temperature or altitude. These dynamic factors can influence the user's movement patterns and effort output and can be considered by the AI model to improve the accuracy and personalization of exercise recognition and performance evaluation.

At 1006, the first data is transformed into second data representing one or more motion features. This transformation can be performed by the AI model, which can analyze the raw motion data to identify motion patterns indicative of specific physical activities. In some embodiments, the AI model can detect recurring acceleration signatures, directional changes, or rhythmic movement sequences that correspond to particular exercise motions. In some embodiments, the AI model may divide the first data into time-based segments, such as one-second intervals or segments of another duration, and determine a motion feature corresponding to each segment. These motion features can include statistical descriptors, frequency-domain characteristics, or learned representations extracted by neural network layers. In some embodiments, the second data can be represented as an array of values, with each value characterizing a motion feature determined for a corresponding segment of the first data. This segmented and feature-rich representation enables downstream components of the system to perform accurate and efficient activity recognition and performance assessment.

At 1008, an exercise activity being performed by the user is predicted based on the first data, the one or more physiological characteristics, and the second data. This prediction can be performed by the AI model. In some embodiments, the AI model can compare the second data representing motion features extracted from raw motion signals with learned motion feature profiles stored in a feature store. These stored profiles can represent known patterns associated with specific exercise activities and can be indexed or weighted based on physiological characteristics to improve prediction accuracy.

In some embodiments, the AI model can also predict additional exercise-related metrics, such as the number of repetitions performed during the activity and/or the amount of weight or resistance used by the user. In some embodiments, the AI model can be configured to derive these predictions from features such as temporal patterns, motion intensity, and biomechanical cues present in the motion data. In some embodiments, the AI model can use historical exercise-related data from past workout sessions (e.g., an amount of weight or resistance used by the user during their most recent performance of an exercise activity) to predict current exercise-related metrics.

As described, the AI model can incorporate a large number (e.g., millions) of parameters, such as weights, biases, and nodes. The large scale of the model can enable the model to efficiently identify intricate patterns in the user’s motion data and rapidly assess those patterns in view of the user’s unique physiological profile. This architecture can support high-accuracy prediction of exercise activity type across hundreds or thousands of distinct movement types and variations. The AI model can be configured not only to distinguish between different classes of movement (e.g., squats, presses, hinge movements, pulling movements, rotational movements, etc.) but also to differentiate between sub-types of exercises. For example, the model can distinguish between bench presses performed with different grip configurations (e.g., wide grip vs. narrow grip), or between squats performed with different types of weights (e.g., barbell squats vs. kettlebell squats vs. dumbbell squats). In some embodiments, the model can determine whether the user is performing an exercise using a machine or free weights, based on motion constraints and movement signatures. In some embodiments, predicting the exercise activity can include assigning a probability value to each possible exercise activity. Probability values can be assigned using any suitable technique, for example, using a softmax function that converts raw model outputs into a normalized probability distribution. The probability value assigned to each exercise activity can represent the likelihood that the user is performing that exercise activity. In some embodiments, these probability values can be compared against a predefined threshold. If the probability value associated with a particular exercise activity exceeds the threshold, the model can classify that activity as a valid prediction. This probabilistic approach can the system to handle uncertainty in motion data and supports flexible decision-making in real-time exercise recognition.

In some embodiments, data in addition to raw motion data (or data derived therefrom) can be used to improve the accuracy and confidence of exercise activity predictions. For example, environmental signals such as audio, Wi-Fi, Bluetooth, GPS, and magnetic field strength signals can be monitored and used to provide additional contextual information regarding the user’s motion. This integration of motion data with environmental and contextual information enables more accurate and personalized exercise recognition.

In some embodiments, wireless network signals can be used to infer the user’s location within a fitness facility. Over time, associations between these signals and specific areas of the gym can be learned. When the AI model detects an exercise but is uncertain between similar activities (e.g., a kettlebell swing versus a dumbbell swing), the model can leverage location-based context to increase confidence in its prediction.

In some embodiments, magnetic field signals can be used to detect movement of metal objects in a vicinity of the user relative to the user. The AI model can leverage such signals to determine, for example, how the user is moving a set of weights. When the AI model detects an exercise but is uncertain between similar activities, the model can leverage magnetic field-based context to increase confidence in its prediction.

In some embodiments, audio signals detected by, e.g., a microphone in a set of wireless headphones or other wearable device, can be used to detect sound (e.g., a sound produced by a weight coming into contact with the ground) in a vicinity of the user. Over time, associations between these signals and specific exercise activities can be learned. When the AI model detects an exercise but is uncertain between similar activities, the model can leverage audio-based context to increase confidence in its prediction.

In some embodiments, the AI model can detect compound exercises that involve multiple, distinct types of motion. The AI model may identify distinct signatures, features, or patterns within the first data that are indicative of the user performing a single exercise activity that includes two or more distinguishable movement types. For example, if the user is performing a compound exercise activity that includes both a squat and a bicep curl, the AI mode may identify a first subset of the first data that corresponds to the squat and a second subset of the first data that corresponds to the bicep curl. The AI model can then predict that the user is performing a compound exercise activity that includes both the squat and the bicep curl.

At 1010, third data characterizing the predicted exercise activity predicted can be provided. Providing the third data can involve transmitting the data over a wireless communication network (e.g., Bluetooth, Wi-Fi, or cellular) to one or more computing devices of the fitness tracking system, such as a wearable computing device (e.g., a smart watch) or a mobile computing device (e.g., a smart phone). In some embodiments, the third data (or a visual representation thereof) can be displayed on a graphical user interface (GUI) of an application associated with the fitness tracking system. The GUI can present the exercise type alongside other relevant metrics, such as repetition count or effort level, on the display of the wearable device, the mobile device, and/or another computing device connected to the system.

In some embodiments, the third data can be provided together with a request for user input confirming whether the predicted exercise activity is accurate. If the user indicates that the prediction is correct, the system can be configured to prompt the user to confirm whether additional metrics associated with the exercise activity (e.g., repetition count or weight/resistance amount) are accurate. If the user indicates that the predicted exercise activity is inaccurate, the system can provide alternative predictions, such as the second-most likely and third-most likely exercise activities determined by the AI model. The AI model can be configured to use such user feedback to improve future exercise activity predictions through retraining or adjustment of model parameters, thereby enhancing accuracy and personalization over time.

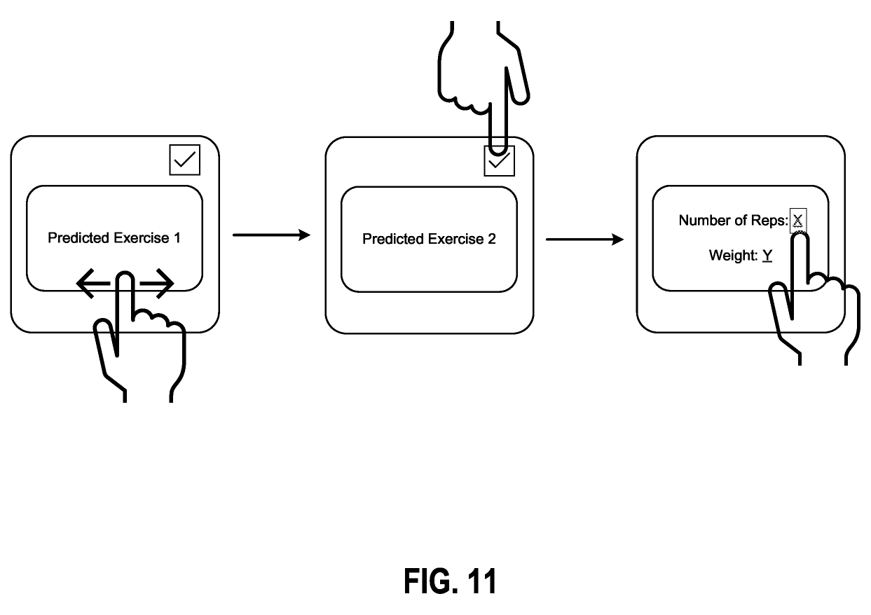

FIG. 11 illustrates views 1100A-1100C of an example GUI of a smart watch application associated with a fitness tracking system.

As shown in view 1100A, the GUI can be configured to display information indicating one or more predicted exercise activities. This display can occur while the user is performing the exercise or immediately after the user completes the exercise, for example, within approximately three seconds of the user finishing a set. The most probable exercise activity, as determined by the AI model implemented by the fitness tracking system, can be displayed first to streamline user confirmation. As shown, the GUI can allow the user to view alternative exercise activity predictions by performing a gesture on the touchscreen display of the smart watch, such as swiping left or right, enabling quick access to, for example, the second-most and third-most likely predictions.

The user can provide input confirming the correct predicted exercise activity by interacting with the GUI, for example, by selecting an icon or button displayed alongside the predicted exercise type, as shown in view 1100B. This interaction can enable the system to validate its prediction and

proceed with displaying additional exercise-related metrics. The confirmation input can be captured through touch gestures, voice commands, or other input mechanisms supported by the smart watch.

As shown in view 1100C, once the user confirms the exercise activity, the GUI can display predicted metrics associated with the exercise, such as the number of repetitions performed, the amount of weight or resistance used, and the estimated effort level. The user can be prompted to verify the accuracy of these metrics and, if necessary, adjust them directly within the GUI. For example, the user may edit the repetition count or input the correct weight used during the exercise. This interaction can ensure that the fitness tracking system maintains accurate records while enabling the AI model to incorporate user feedback for improving future predictions.

The term “connected” or "coupled to" may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references may be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

What is claimed is:

1. A system comprising:
at least one processor; and
non-transitory memory storing instructions which, when executed by the at least one processor, cause the at least one processor to perform operations comprising:
receiving, from one or more wearable devices comprising one or more sensors configured to detect motion of a user, first data characterizing raw motion of the user;
determining, using an artificial intelligence (AI) model, one or more physiological characteristics of the user;
predicting, using the AI model, based on the one or more physiological characteristics and the first data, an exercise activity being performed by the user; and
providing data characterizing the predicted exercise activity.

2. The system of claim 1, wherein the one or more wearable devices comprises a smart watch, a fitness tracking band, wireless headphones, wireless earphones, or a combination thereof.

3. The system of claim 1, wherein the first data comprises a time series of acceleration vectors representing a magnitude and direction of an acceleration of a body part of the user.

4. The system of claim 1, wherein the AI model comprises one or more convolutional neural network (CNN) layers, one or more long short-term memory (LSTM) layers, one or more embedding layers, or a combination thereof.

5. The system of claim 1, wherein the operations further comprise:
transforming, using the AI model, the first data into second data representing one or more motion features.

6. The system of claim 5, wherein transforming the first data into the second data comprises extracting the one or more motion features from the first data by one or more convolutional neural networks (CNNs) of the AI model.

7. The system of claim 1, wherein the operations further comprise:
encoding, by one or more embedding layers of the AI model, the one or more physiological characteristics as one or more values.

8. The system of claim 1, wherein the first data comprises:
data characterizing raw motion of the user during a first time period prior to performing an exercise movement;
data characterizing raw motion of the user during a second time period in which the exercise movement is performed;
data characterizing raw motion of the user during a third time period following performance of the exercise movement.

9. The system of claim 1, wherein the operations further comprise:
predicting, by the AI model, one or more metrics associated with the exercise activity.

10. The system of claim 9, wherein the one or more metrics comprises a first value corresponding to a number of repetitions of the exercise activity and a second value quantifying an amount of weight or resistance used during the exercise activity.

11. The system of claim 1, wherein predicting the exercise activity performed by the user comprises:
identifying a first type of motion based on a first subset of the first data;
identifying a second type of motion based on a second subset of the first data;
predicting a compound exercise activity performed by the user, wherein the compound exercise activity includes the first type of motion and the second type of motion.

12. A method comprising:
receiving, from one or more wearable devices comprising one or more sensors configured to detect motion of a user, first data characterizing raw motion of the user;
determining, using an artificial intelligence (AI) model, one or more physiological characteristics of the user;
predicting, using the AI model, based on the one or more physiological characteristics and the first data, an exercise activity being performed by the user; and
providing data characterizing the predicted exercise activity.

13. The method of claim 12, wherein the first data comprises a time series of acceleration vectors representing a magnitude and direction of an acceleration of a body part of the user.

14. The method of claim 12, further comprising: transforming, using the AI model, the first data into second data representing one or more motion features.

15. The method of claim 12, further comprising:
receiving data characterizing an environment of the user;
wherein predicting the exercise activity is further based on the data characterizing the environment of the user.

16. The method of claim 15, wherein the data characterizing the environment of the user comprises data characterizing an audible noise, a magnetic field strength, or a wireless network signal in a vicinity of the user.

17. The method of claim 15, further comprising:
determining, by the AI model, a location of the user based on the data characterizing the environment of the user;
wherein predicting the exercise activity is further based on the location of the user.

18. The method of claim 12, wherein the operations further comprise:
predicting, by the AI model, one or more metrics associated with the exercise activity.

19. The method of claim 18, wherein the one or more metrics comprise a first value corresponding to a number of repetitions of the exercise activity and a second value quantifying an amount of weight or resistance used during the exercise activity.

20. A non-transitory computer readable storage medium storing instructions which, when executed by at least one processor, cause the at least one processor to perform operations comprising:
receiving, from one or more wearable devices comprising one or more sensors configured to detect motion of a user, first data characterizing raw motion of the user;
determining, using an artificial intelligence (AI) model, one or more physiological characteristics of the user;
predicting, using the AI model, based on the one or more physiological characteristics and the first data, an exercise activity being performed by the user; and
providing data characterizing the predicted exercise activity.
